SEQUENCE LISTING (5 sequences)

SEQ ID NO: 1 - RNA, 22 nt, Homo sapiens
ggguggggauuuguugcauuac

SEQ ID NO: 2 - RNA, 20 nt, Homo sapiens
guguguggaaaugcuucugc

SEQ ID NO: 3 - RNA, 23 nt, Homo sapiens
ugagugugugugugugagugugu

SEQ ID NO: 4 - RNA, 106 nt, Homo sapiens
gugcucgcuucggcagcacauauacuaaaauuggaacgauacagagaagauuagcauggccccugcgcaaggaugacacgcaaauucgugaagcguuccauauuuu

SEQ ID NO: 5 - RNA, 121 nt, Homo sapiens
uuuacggccacaccacccugaacgcacgggaucucgacugaccuugaaagcuaagcagagucgggccugguuaguacuugggaugcgagacccccugggaauacagggugcugaaggcuuu

(19) United States
(12) Patent Application Publication
(10) Pub. No.: US 2011/0171323 A1
(43) Pub. Date: Jul. 14, 2011

(54) METHODS AND KITS TO PREDICT PROGNOSTIC AND THERAPEUTIC OUTCOME IN SMALL CELL LUNG CANCER

(75) Inventor: GLEN J. WEISS, Phoenix, AZ (US)

(73) Assignees: THE TRANSLATIONAL GENOMICS RESEARCH INSTITUTE, Phoenix, AZ (US); SCOTTSDALE HEALTHCARE, Scottsdale, AZ (US)

(21) Appl. No.: 12/987,093

(22) Filed: Jan. 8, 2011

Related U.S. Application Data
(60) Provisional application No. 61/293,634, filed on Jan. 9, 2010.

(51) Int. Cl. A61K 33/24; G01N 33/574; C12Q 1/68; C40B 30/04; A61K 31/282; A61K 31/366; A61K 31/4375; A61K 31/675; A61K 31/704; A61K 31/437; A61P 35/00

(52) U.S. Cl. 424/649; 436/501; 506/9; 514/492; 514/452; 514/283; 514/110; 514/34; 514/282; 435/6.14

(57) ABSTRACT

Disclosed herein are methods used in the identification of cancer patients likely or unlikely to respond to systemic chemotherapy, methods of treating cancer patients based upon the identification, and kits that facilitate the identification. The methods and kits involve detecting the expression of specific microRNA.

### PRIORITY CLAIM

This application claims priority under 35 US §119(e) from U.S. Provisional Application 61/293,634, filed on 9 Jan. 2010, entitled METHODS AND KITS TO PREDICT PROGNOSTIC AND THERAPEUTIC OUTCOME IN SMALL CELL LUNG CANCER, which is hereby incorporated by reference in its entirety.

### FIELD OF THE INVENTION

The invention is related to tests that use biomarkers to predict disease outcome. More particularly, the invention is related to tests that use microRNA biomarkers to predict whether or not a cancer patient will be resistant to chemotherapy.

### BACKGROUND OF THE INVENTION

Lung cancer is by far the leading cause of cancer-related deaths in the United States. There is an estimated 159,390 deaths from lung cancer in 2009, accounting for around 29% of all cancer cases detected. The most prevalent types of lung cancer are classified histologically as non-small cell lung cancer (NSCLC) and small cell lung cancer (SCLC). SCLC makes up approximately 15% of lung cancers diagnosed. In 2009, over 32,000 new SCLC cases were diagnosed in the United States alone. Jemal A et al, CA Cancer J Clin 59, 225-249 (2009). SCLC has a very aggressive course, with approximately 60-70% of patients having extensive-stage disease at the time of diagnosis. Jackman D M and Johnson B E, Lancet 366, 1385-1396 (2005). Nearly 30 years ago, platinum-based chemotherapy was used in SCLC treatment and remains the backbone of current combination strategies. While the majority of SCLC patients respond to initial systemic chemotherapy, those with disease progression at first response assessment (chemoresistance) have inferior outcomes. Sandler A B, Semin Oncol 30, 9-25 (2003).

Identifying patients with chemoresistant cancer prior to treatment is important to both the clinician and patients. Patients with chemoresistant cancer could be identified and placed on an alternative form of treatment. This spares patients from suffering the debilitating side effects of systemic chemotherapy for little to no benefit. Identifying patients with chemoresistant cancer prior to treatment becomes even more important as new targeted, personalized medicine based therapeutics become available and greatly facilitates clinical trials for these new therapeutics.

Because standard clinical and laboratory parameters fail to identify patients with chemoresistant cancer, molecular biomarkers might be sought. There are several methods available to find biomarkers that associate with disease outcome. Genome wide association screening (GWAS) provides a set of germline mutations that are potentially predictive of outcome. Gene expression profiling provides a signature of genes, the expression of which is associated with prognosis. A major limitation with these approaches is that cellular phenotype is ultimately defined by protein expression. Often, protein expression shows only a limited correlation with mutations in genomic DNA or messenger RNA (mRNA expression.) This is due to the fact that mRNA expression is also regulated by many events including translational regulation. As a result, no biomarker predictive of chemoresistance has been successfully put into use by clinicians treating lung cancer to date.

MicroRNAs (also known as miRs or miRNAs, among other names) are a class of small non-coding RNAs, often between 20 to 22 nucleotides in length. Calin G A et al, Proc Natl Acad Sci USA 99, 15524-15529 (2002). These RNA molecules post-transcriptionally regulate gene expression by binding to complementary sequences in the 3′ untranslated region (3′UTR) of the target mRNA This can ultimately lead to translational repression or mRNA cleavage depending on the degree of sequence complementarity between the miRNA and the target mRNA. Nelson K M and Weiss G J Mol Cancer Ther 7, 3655-3660 (2008). Either of these result in a repression of protein translation. Because the miRNA binding sequences in the 3′UTRs of the target mRNAs are often highly conserved, the expression of a single miRNA has the potential to regulate multiple proteins and potentially entire protein pathways. Experimentally, dysregulation of miRNAs has been shown lead to malignant progression in cells. Calin G A et al, Proc Natl Acad Sci USA 101, 11755-11760 (2004). Furthermore, miRNA expression patterns can potentially classify human cancers with fewer discriminators than gene expression arrays. Lu J et al, Nature 435, 834-838 (2005). MiRNA microarray analysis has been used to identify miRNA expression profiles capable of discriminating lung cancers from non-cancerous lung tissues. Yanihara N et al Cancer Cell 9, 189-198 (2006). MiRNA microarray analysis has also been used to show miRNA differences in tumor histology. Miko E et al, Exp Lung Res 35, 646-664 (2009).

In summary, miRNAs can be powerful predictive biomarkers. Methods that use of reagents capable of detecting miRNA that are capable of classifying lung cancer patients into those likely to have chemoresistant cancer and those unlikely to have chemoresistant cancer prior to treatment would be powerful tools in the staging of lung cancer patients and in the subsequent, treatment of those patients.

### BRIEF SUMMARY OF THE INVENTION

The present invention provides, among other things, a method that allows the classification of SCLC patients into cohorts. More specifically, the present invention facilitates the identification of a cohort of patients least to respond to systemic chemotherapy. The cohort of patients unlikely to respond to systemic chemotherapy is identified on the basis of the expression of microRNA such as miR-92a-2* (SEQ ID NO. 1), miR-147 (SEQ ID NO. 2) or miR-585.

In one embodiment of the invention; a first oligonucleotide capable of binding to a first biomarker is added to a mixture comprising a nucleic acid isolated from a sample from a patient. The mixture may be subjected to conditions that allow detection of the binding of the first oligonucleotide to the biomarker. The patient may be classified the patient into a cohort on the basis of the binding of the first oligonucleotide to the nucleic acid isolated from the sample. The biomarker may be any of miR-92a-2*, miR-147, or miR-585, either alone or in combination with each other or with any other biomarker. The cohort may be a cohort of patients that is likely to respond to systemic chemotherapy or a cohort of patients unlikely to respond to systemic chemotherapy. The sample may be any sample such as a sample from a blood fraction such as serum, plasma, or whole blood or the sample may include tissue obtained from a tumor. The patient may be suspected of having small-cell lung cancer, such as a patient that is known to have small-cell lung cancer. The first oligonucleotide may be any type of oligonucleotide, including a stem-loop oligonucleotide. The method may further comprise the addition of a reverse transcriptase to the mixture, in which case the conditions may further comprise synthesis of a double stranded reverse transcription product comprising the biomarker. Should the conditions comprise the formation of a reverse transcription product comprising the biomarker, the method may further comprise adding a second oligonucleotide and a third oligonucleotide to the mixture. The second oligonucleotide and the third oligonucleotide would both have a sequence that would render it capable of binding to some part of the reverse transcription product and each would bind to opposite strands of the reverse transcription product. Should second and third oligonucleotides be added to the mixture, the conditions may further comprise nucleic acid amplification such as polymerase chain reaction. Should the method comprise nucleic acid amplification, then the method may further comprise adding a fourth oligonucleotide to the mixture. Any fourth oligonucleotide would have a sequence that renders it capable of binding to a sequence on the reverse transcription product between the sequences to which the second oligonucleotide and the third oligonucleotide are capable of binding. Any fourth oligonucleotide may comprise a fluorescent label. The fluorescent label may be any fluorescent label such as FAM, dR110, 5-FAM, 6FAM, dR6G, JOE, HEX, VIC, TET, dTAMRA, TAMRA, NED, dROX, PET, BHQ+, Gold540, or LIZ. In other aspects of the invention, DNA sequencing may be performed on the reverse transcription product. In other aspects of the invention, the first oligonucleotide may be affixed to a substrate. The substrate may be any substrate. Should the first oligonucleotide be affixed to a substrate, then a second oligonucleotide may also be affixed to the substrate so as to form a microarray. The result used to classify the patient into the cohort may be any result that detects the binding of the reagent to the biomarker. One example of such a result is expression of the biomarker below a previously determined threshold. An example of a member of a cohort of patients likely to respond to systemic chemotherapy is a patient that may be predicted to survive for about 270 days following systemic chemotherapy. An example of a member of a cohort of patients unlikely to respond to systemic chemotherapy is a patient that may be predicted to survive for about 70 days following systemic chemotherapy.

In another embodiment of the invention, a first oligonucleotide capable of binding to a first biomarker is added to a mixture comprising a nucleic acid isolated from a sample from a patient. The mixture is subjected to conditions that allow detection of the binding of the first oligonucleotide to the biomarker. The patient is treated on the basis of the binding of the first oligonucleotide to the nucleic acid isolated from the sample. The biomarker may be any of miR-92a-2*, miR-147, or miR-585, either alone or in combination with each other or with any other biomarker. The result may be any result, including expression of the biomarker below a predetermined threshold. Should the result be expression of the biomarker below the predetermined threshold, then treatment of the patient may comprise administration of systemic chemotherapy. The predetermined threshold may be any threshold. One example is an expression level of 0.5 as measured by quantitative reverse transcription PCR normalized to the expression of SEQ ID NO. 4 and SEQ ID NO. 5. In this case, the threshold may be a value less than 0.5, such as 0.3. Should systemic chemotherapy be administered, then the treatment may comprise administration of one or more of the following: cisplatin, carboplatin, etoposide, ironectan, topotecan, cyclophosphamide, doxorubicin, vincristine, amrubicin, epirubicin, or S-1. In another aspect of the invention, the result comprises expression of the biomarker above a threshold. Should the result comprise expression above a threshold, then treating the patient may comprise administration of a pharmaceutical composition with an effect on a chemoresistant tumor. In this aspect, the threshold may be any threshold such as an expression level of 0.2 measured by quantitative reverse transcription PCR normalized to the expression of RNU-6 (SEQ ID NO. 4) or 5S-rRNA (SEQ ID NO. 5.) In this case, the threshold may comprise an expression level of 0.5. Should the treatment comprise administration of a pharmaceutical composition with an effect on a chemoresistant tumor, then the pharmaceutical composition may comprise one or more of the following: CD9 inhibitors, chemokine CXCL12 agonists, fibronectin β1 integrin inhibitors, FGFR inhibitors, xc-cysteine transporter inhibitors, urokinase plasminogen activator (uPA) inhibitors, and ATP binding cassette (ABC) transporter inhibitors.

In another embodiment of the invention, a kit comprising a first oligonucleotide capable of binding to a first biomarker that may be any of miR-92a-2*, miR-147, or miR-585 and an indication of a result of the binding of the first biomarker to a nucleic acid isolated from a sample is assembled. The result may be a result that signifies the patient as belonging to one of the following cohorts: a cohort of patients likely to respond to systemic chemotherapy and a cohort of patients unlikely to respond to systemic chemotherapy. The first oligonucleotide may be any oligonucleotide such as a stem loop oligonucleotide. In one aspect of the invention, the kit may comprise a second oligonucleotide wherein the second oligonucleotide is capable of binding to a second biomarker and wherein the second biomarker comprises a housekeeping gene. The second biomarker may include RNU-6 (SEQ ID NO. 4) or 5s-rRNA (SEQ ID NO. 5). In other aspects of the invention, the kit may comprise an enzyme. The enzyme may be any enzyme including a DNA polymerase, a thermostable DNA polymerase such as Taq or Pfu, or a reverse transcriptase. In some aspects of the invention, the first oligonucleotide is affixed to a substrate. Should the first oligonucleotide be affixed to a substrate, then the kit may further comprise a second oligonucleotide affixed to the solid substrate configured to form a microarray. The indication may be any indication of a result that signifies binding of the reagent to the biomarker. Examples include a positive control, a numerical value, a Ct value, or a level of expression normalized to a housekeeping gene. The indication may comprise software configured to detect a level of expression as input and classification of the subject into the cohort as output. The indication may comprise a writing. The writing may be any writing, including a writing that is physically included with the kit or a writing that is made available through a website.

It is an object of the invention to stage SCLC patients with regard to the likelihood that they will respond to systemic chemotherapy in order to better inform treatment.

It is an object of the invention to provide alternative therapies for SCLC patients unlikely to respond to systemic chemotherapy.

It is an object of the invention to provide a test that rapidly predicts which patients are and are not likely to respond to systemic chemotherapy.

It is an object of the invention to provide kits that facilitate the performance of a test that rapidly predicts which patients are and are not likely to respond to systemic chemotherapy.

### BRIEF DESCRIPTION OF THE FIGURES

A more complete understanding of the present invention may be derived by referring to the detailed description when considered in connection with the following figures.

FIG. 1 depicts the survival by miR-92a-2* expression. The lighter line depicts the survival curve for patients with miR-92a-2* expression levels <0.24 (normalized to RNU6 and 5S-rRNA), and the red line depicts survival curve for patients with miR-92a-2* expression levels >0.24 (normalized to RNU6 and 5S-rRNA). The survival curves were found to be significantly different with a log-rank p-value of 0.0001.

FIG. 2 illustrates that overexpression of miR-92a-2* causes greater expression HGF protein relative to controls in H526 cells.

FIG. 3 illustrates that overexpression of miR-92a-2* causes increased phosphorylation of c-MET in H526 cells.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. Aspects and applications of the invention presented here are described below in the figures and detailed description of the invention. Unless specifically noted, it is intended that the words and phrases in the specification and the claims be given their plain, ordinary, and accustomed meaning to those of ordinary skill in the applicable arts.

The use of the words “function,” “means” or “step” in the Detailed Description or Description of the Figures or claims is not intended to somehow indicate a desire to invoke the special provisions of 35 U.S.C. §112, ¶ 6, to define the invention. To the contrary, if the provisions of 35 U.S.C. §112, ¶ 6 are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function.

All publications and/or patents mentioned herein are hereby incorporated by reference in their entireties. The publications and patents disclosed herein are provided solely for illustrating the state of the art and enabling the invention. Nothing herein is to be construed as an admission that the inventors are not entitled to antedate any publication and/or patent including any publication and/or patent cited herein.

The invention encompasses methods of using a reagent capable of binding to a biomarker to produce a result that may be used to classify a lung cancer patient into a cohort. Specifically, the reagent may be any reagent that binds to one or more of the following biomarkers: miR-92a-2* (SEQ ID NO. 1), miR-147 (SEQ ID NO. 2), or miR-585 (SEQ ID NO. 3). The result may be any result that signifies binding of the reagent to the biomarker. The cohort in which the patient might be classified may be (a) a cohort of patients likely to respond to systemic chemotherapy, or (b) a cohort of patients unlikely to respond to systemic chemotherapy.

The invention further encompasses methods of predicting whether or not a patient will respond to systemic chemotherapy and treating the patient on the basis of that prediction.

The invention further encompasses kits that facilitate the performance of these methods.

A biomarker may be any molecular structure produced by a cell, expressed inside the cell, accessible on the cell surface, or secreted by the cell. A biomarker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination. A biomarker may also be called a target and the terms may be used interchangeably.

A biomarker may be represented by the sequence of a nucleic acid from which it can be derived. Examples of such nucleic acids include miRNA, tRNA, siRNA, mRNA, cDNA, or genomic DNA sequences. While a biomarker may be represented by the sequence of a single nucleic acid strand (e.g. 5′→3′), nucleic acid reagents that bind the biomarker may also bind to the complementary strand (e.g. 3′→5′). A biomarker also encompasses the reverse transcription product of an RNA molecule. Alternatively, a biomarker may be represented by a protein sequence. The concept of a biomarker is not limited to the products of the exact nucleic acid sequence or protein sequence by which it may be represented. Rather, a biomarker encompasses all molecules that may be detected by a method of assessing the expression of the biomarker.

Examples of molecules encompassed by a biomarker include point mutations, silent mutations, deletions, frameshift mutations, translocations, alternative splicing derivatives, differentially methylated sequences, differentially modified protein sequences, truncations, soluble forms of cell membrane associated biomarkers, and any other variation that results in a product that may be identified as the biomarker. The following nonlimiting examples are included for the purposes of clarifying this concept: If expression of a specific biomarker in a sample is assessed by RTPCR, and if the sample expresses an mRNA sequence different from the sequence used to identify the specific biomarker by one or more nucleotides, but the biomarker may still be detected using RTPCR, then the specific biomarker encompasses the sequence present in the sample. Alternatively if expression of a specific biomarker in a sample is assessed by an antibody and the amino acid sequence of the biomarker in the sample differs from a sequence used to identify biomarker by one or more amino acids, but the antibody is still able to bind to the version of the biomarker in the sample, then the specific biomarker encompasses the sequence present in the sample.

Expression encompasses any and all processes through which material derived from a nucleic acid template may be produced. Expression thus includes processes such as RNA transcription, mRNA splicing, protein translation, protein folding, post-translational modification, membrane transport, associations with other molecules, addition of carbohydrate moeties to proteins, phosphorylation, protein complex formation and any other process along a continuum that results in biological material derived from genetic material whether in vitro, in vivo, or ex vivo. Expression also encompasses all processes through which the production of material derived from a nucleic acid template may be actively or passively suppressed. Such processes include all aspects of transcriptional and translational regulation. Examples include heterochromatic silencing, differential methylation, transcription factor inhibition, any form of RNAi silencing, microRNA silencing, alternative splicing, protease digestion, posttranslational modification, and alternative protein folding.

Expression may be assessed by any number of methods used to detect material derived from a nucleic acid template used currently in the art and yet to be developed. Examples of such methods include any nucleic acid detection method including the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing, direct sequencing of genomic DNA, or any other method of detecting a specific nucleic acid now known or yet to be disclosed. Other examples include any process of assessing protein expression including, for example; flow cytometry, immunohistochemistry, ELISA, Western blot, and immunoaffinity chromatography, HPLC, mass spectrometry, protein microarray analysis, PAGE analysis, isoelectric focusing, 2-D gel electrophoresis, or any enzymatic assay. Methods of detecting expression may include methods of purifying nucleic acid, protein, or some other material depending on the type of biomarker. Any method of nucleic acid purification may be used, depending on the type of biomarker. Examples include phenol alcohol extraction, ethanol extraction, guanidium isothionate extraction, gel purification, size exclusion chromatography, cesium chloride preparations, and silica resin preparation. Any method of protein purification may be used, also depending on the type of biomarker. Examples include size exclusion chromatography, hydrophobic interaction chromatography, ion exchange chromatography, affinity chromatography (including affinity chromatography of tagged proteins), metal binding, immunoaffinity chromatography, and HPLC.

Nucleic acids may be isolated by any process that purifies nucleic acid from a sample. Nucleic acid isolation procedures may include phenol-chloroform extraction, alcohol precipitation, binding to glass or synthetic beads, cesium chloride purification, gel purification, or any other method that results in a greater proportion of nucleic acid relative to other components than was present in the original sample now known or yet to be discovered. Nucleic acid isolation procedures may be used alone or in combination with each other or with other procedures not mentioned herein.

Nucleic acid amplification is a process by which copies of a nucleic acid may be made from a source nucleic acid. Nucleic acids that may be subjected to amplification may be from any source. In some nucleic amplification methods, the copies are generated exponentially. Examples of nucleic acid amplification include but are not limited to: the polymerase chain reaction (PCR), ligase chain reaction (LCR,) self-sustained sequence replication (3SR), nucleic acid sequence based amplification (NASBA,) strand displacement amplification (SDA,) amplification with Qβ replicase, whole genome amplification with enzymes such as φ29, whole genome PCR, in vitro transcription with any RNA polymerase, or any other method by which copies of a desired sequence are generated.

Polymerase chain reaction (PCR) is a particular method of amplifying DNA, generally involving the mixing of a nucleic sample, two or more oligonucleotide primers, a DNA polymerase, which may be a thermostable DNA polymerase such as Taq or Pfu, and deoxyribose nucleoside triphosphates (dNTP's). In general, the reaction mixture is subjected to temperature cycles comprising a denaturation stage, (typically 80-100° C.) an annealing stage with a temperature that is selected based on the melting temperature (Tm) of the primers and the degeneracy of the primers, and an extension stage (for example 40-75° C.) In real-time PCR analysis, additional reagents, methods, optical detection systems, oligonucleotide probes and devices may be used that allow a measurement of the magnitude of fluorescence in proportion to concentration of amplified DNA. In such analyses, incorporation of fluorescent dye into the amplified strands may be detected or labeled oligonucleotide probes that bind to a specific sequence during the annealing phase release their fluorescent tags during the extension phase. Either of these will allow a quantification of the amount of specific DNA present in the initial sample. Often, the result of a real-time PCR will be expressed in the terms of cycle threshold (Ct) values. The Ct represents the number of PCR cycles for the fluorescent signal from a real-time PCR reaction to cross a threshold value of fluorescence. Ct is inversely proportional to the amount of target nucleic acid originally present in the sample. RNA may be detected by PCR analysis by creating a DNA template from RNA through a reverse transcriptase enzyme.

Other methods used to assess expression include the use of natural or artificial reagents or ligands capable of specifically binding a biomarker. Such reagents include antibodies, antibody complexes, conjugates, natural ligands, small molecules, nanoparticles, oligonucleotides or other nucleic acid reagents or any other molecular entity capable of specific binding to a biomarker. Antibodies may be monoclonal, polyclonal, or any antibody fragment including a Fab, F(ab)₂, Fv, scFv, phage display antibody, peptibody, multispecific ligand, or any other reagent with specific binding to a biomarker. Reagents may be associated with a label such as a radioactive isotope or chelate thereof, dye (fluorescent or nonfluorescent,) stain, enzyme, metal, or any other substance capable of aiding a machine or a human eye from differentiating a cell expressing a biomarker from a cell not expressing a biomarker. Additionally, expression may be assessed by reagents associated with substances capable of killing the cell. Such substances include protein or small molecule toxins, cytokines, pro-apoptotic substances, pore forming substances, radioactive isotopes, or any other substance capable of killing a cell.

A reagent may be added to a mixture by any of a number of methods, depending on (for example) its formulation, concentration and the amount to be added. These methods include manual pipetting, addition of a solid form of the reagent, use of an automated system, or any other method by which one or more materials may be added to a mixture. Addition of a reagent to a mixture also encompasses addition of a mixture containing a sample to a reagent that is affixed to a substrate such as in a blot or array configuration.

Differential expression encompasses any detectable difference between the expression of a biomarker in one sample relative to the expression of the biomarker in another sample. Differential expression may be assessed by a detector, an instrument containing a detector, or by aided or unaided human eye. Examples include but are not limited to differential staining of cells in an IHC assay configured to detect a biomarker, differential detection of bound RNA on a microarray to which a sequence capable of binding to the biomarker is bound, differential results in measuring RTPCR measured in the number of PCR cycles necessary to reach a particular optical density at a wavelength at which a double stranded DNA binding dye (e.g. SYBR Green) incorporates, differential results in measuring label from a reporter probe used in a real-time RTPCR reaction, differential detection of fluorescence on cells using a flow cytometer, differential intensities of bands in a Northern blot, differential intensities of bands in an RNAse protection assay, differential cell death measured by apoptotic biomarkers, differential cell death measured by shrinkage of a tumor, or any method that allows a detection of a difference in signal between one sample or set of samples and another sample or set of samples.

The expression of the biomarker in a sample may be compared to a level of expression predetermined to predict the presence or absence of a particular physiological characteristic. The level of expression may be derived from a single control or a set of controls. A control may be any sample with a previously determined level of expression. A control may comprise material within the sample or material from sources other than the sample. Alternatively, the expression of a biomarker in a sample may be compared to a control that has a level of expression predetermined to signal or not signal a cellular or physiological characteristic. This level of expression may be derived from a single source of material including the sample itself or from a set of sources. Comparison of the expression of the biomarker in the sample to a particular level of expression results in a prediction that the sample exhibits or does not exhibit the cellular or physiological characteristic.

Prediction of a cellular or physiological characteristic includes the prediction of any cellular or physiological state that may be predicted by assessing the expression of a biomarker. Examples include the identity of a cell as a particular cell including a particular normal or cancer or other disease cell type, the likelihood that one or more diseases is present or absent, the likelihood that a present disease will progress, remain unchanged, or regress, the likelihood that a disease will respond or not respond to a particular therapy, or any other outcome. Further examples include the likelihood that a cell will move, senesce, apoptose, differentiate, metastasize, or change from any state to any other state or maintain its current state.

The expression of a biomarker in a sample may be used to classify a subject, such as a patient, into one or more cohorts. A cohort may comprise one or more subjects, wherein each subject in the cohort may have one or more characteristics in common. Alternatively, each subject in the cohort may be similar to other members of the cohort with regard to a particular characteristic—especially when the characteristic may be expressed by a numerical value. A characteristic may be any characteristic such as a level of expression or a cellular or physiological characteristic.

Expression of a biomarker in a sample may be more or less than that of a level predetermined to predict the presence or absence of a cellular or physiological characteristic. The expression of the biomarker in the sample may be more than 1,000,000×, 100,000×, 10,000×, 1000×, 100×, 10×, 5×, 2×, 1×, 0.5×, 0.1× 0.01×, 0.001×, 0.0001×, 0.00001×, 0.000001×, 0.0000001× or less than that of a level predetermined to predict the presence or absence of a cellular or physiological characteristic.

Expression of a biomarker may be compared to that of a housekeeping gene in order to account for experimental variability such as sample loading. A housekeeping gene may be any constitutively active gene, the expression of which is constant and generally independent of cellular state. Examples of housekeeping genes include HSP90, β-actin, and various t-RNA or r-RNAs. One skilled in the art would understand how to use the expression of a housekeeping gene to normalize expression of a biomarker and would understand how to select the proper housekeeping gene used to normalize the expression of any given biomarker.

The invention contemplates assessing the expression of at least one biomarker in any biological sample from which the expression may be assessed. One skilled in the art would know to select a particular biological sample and how to collect said sample depending upon the biomarker that is being assessed. Biological samples include tissue samples derived from biopsy, necropsy or other in vivo or ex vivo collection of any tissue. Examples of tissues include prostate, breast, skin, muscle, fascia, brain, endometrium, lung, head and neck, pancreas, small intestine, blood, liver, testes, ovaries, colon, skin, stomach, esophagus, spleen, lymph node, bone marrow, cellular fraction from blood, kidney, placenta, fetus, or any other component of a living thing. The sample may comprise fluid, semisolid or gel sample, such as peripheral blood, lymph fluid, ascites, serous fluid, pleural effusion, sputum, cerebrospinal fluid, amniotic fluid, lacrimal fluid, stool, urine, or any other material that may be collected from a living thing. Samples may be collected in any of a variety of forms including a fluid sample, single cells, whole organs or any fraction of a whole organ, in any condition including in vitro, ex vivo, in vivo, post-mortem, fresh, fixed (such as in an FFPE tissue or section therefrom), or frozen.

One type of cellular or physiological characteristic is the risk that a particular disease outcome will occur. Assessing this risk includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

Determining the level of expression that signifies a physiological or cellular characteristic may be assessed by any of a number of methods. The skilled artisan will understand that numerous methods may be used to select a level of expression for a particular biomarker or a plurality of biomarkers that signifies a particular physiological or cellular characteristics. In diagnosing the presence of a disease, a threshold value may be obtained by performing the assay method on samples obtained from a population of patients having a certain type of disease (cancer for example,) and from a second population of subjects that do not have the disease. In assessing disease outcome or the effect of treatment, a population of patients, all of which have, a disease such as cancer, may be followed for a period of time. After the period of time expires, the population may be divided into two or more groups. For example, the population may be divided into a first group of patients whose disease progresses to a particular endpoint and a second group of patients whose disease does not progress to the particular endpoint. Examples of endpoints include disease recurrence, death, metastasis or other states to which disease may progress. If expression of the biomarker in a sample is more similar to the predetermined expression of the biomarker in one group relative to the other group, the sample may be assigned a risk of having the same outcome as the patient group to which it is more similar.

In addition, one or more levels of expression of the biomarker may be selected that signify a particular physiological or cellular characteristic. For example, Receiver Operating Characteristic curves, or “ROC” curves, may be calculated by plotting the value of a variable versus its relative frequency in two populations. For any particular biomarker, a distribution of biomarker expression levels for subjects with and without a disease may overlap. This indicates that the test does not absolutely distinguish between the two populations with complete accuracy. The area of overlap indicates where the test cannot distinguish the two groups. A threshold is selected. Expression of the biomarker in the sample above the threshold indicates the sample is similar to one group and expression of the biomarker below the threshold indicates the sample is similar to the other group. The area under the ROC curve is a measure of the probability that the expression correctly indicated the similarity of the sample to the proper group. See, e.g., Hanley et al., Radiology 143: 29-36 (1982) hereby incorporated by reference.

Additionally, levels of expression may be established by assessing the expression of a biomarker in a sample from one patient, assessing the expression of additional samples from the same patient obtained later in time, and comparing the expression of the biomarker from the later samples with the initial sample or samples. This method may be used in the case of biomarkers that indicate, for example, progression or worsening of disease or lack of efficacy of a treatment regimen or remission of a disease or efficacy of a treatment regimen.

Other methods may be used to assess how accurately the expression of a biomarker signifies a particular physiological or cellular characteristic. Such methods include a positive likelihood ratio, negative likelihood ratio, odds ratio, and/or hazard ratio. In the case of a likelihood ratio, the likelihood that the expression of the biomarker would be found in a sample with a particular cellular or physiological characteristic is compared with the likelihood that the expression of the biomarker would be found in a sample lacking the particular cellular or physiological characteristic.

An odds ratio measures effect size and describes the amount of association or non-independence between two groups. An odds ratio is the ratio of the odds of a biomarker being expressed in one set of samples versus the odds of the biomarker being expressed in the other set of samples. An odds ratio of 1 indicates that the event or condition is equally likely to occur in both groups. An odds ratio greater or less than 1 indicates that expression of the biomarker is more likely to occur in one group or the other depending on how the odds ratio calculation was set up.

A hazard ratio may be calculated by estimate of relative risk. Relative risk is the chance that a particular event will take place. It is a ratio of the probability that an event such as development or progression of a disease will occur in samples that exceed a threshold level of expression of a biomarker over the probability that the event will occur in samples that do not exceed a threshold level of expression of a biomarker. Alternatively, a hazard ratio may be calculated by the limit of the number of events per unit time divided by the number at risk as the time interval decreases. In the case of a hazard ratio, a value of 1 indicates that the relative risk is equal in both the first and second groups. A value greater or less than 1 indicates that the risk is greater in one group or another, depending on the inputs into the calculation.

Additionally, multiple threshold levels of expression may be determined. This can be the case in so-called “tertile,” “quartile,” or “quintile” analyses. In these methods, multiple groups can be considered together as a single population, and are divided into 3 or more bins having equal numbers of individuals. The boundary between two of these “bins” may be considered threshold levels of expression indicating a particular level of risk of a disease developing or signifying a physiological or cellular state. A risk may be assigned based on which “bin” a test subject falls into.

A subject includes any human or non-human mammal, including for example: a primate, cow, horse, pig, sheep, goat, dog, cat, or rodent, capable of developing cancer including human patients that are suspected of having cancer, that have been diagnosed with cancer, or that have a family history of cancer. Methods of identifying subjects suspected of having cancer include but are not limited to: physical examination, family medical history, subject medical history including exposure to environmental factors, biopsy, or any of a number of imaging technologies such as ultrasonography, computed tomography, magnetic resonance imaging, magnetic resonance spectroscopy, or positron emission tomography.

Cancer cells include any cells derived from a tumor, neoplasm, cancer, precancer, cell line, malignancy, or any other source of cells that have the potential to expand and grow to an unlimited degree. Cancer cells may be derived from naturally occurring sources or may be artificially created. Cancer cells may also be capable of invasion into other tissues and metastasis. Cancer cells further encompass any malignant cells that have invaded other tissues and/or metastasized. One or more cancer cells in the context of an organism may also be called a cancer, tumor, neoplasm, growth, malignancy, or any other term used in the art to describe cells in a cancerous state.

Examples of cancers that could serve as sources of cancer cells include solid tumors such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Additional cancers that may serve as sources of cancer cells include blood borne cancers such as acute lymphoblastic leukemia (“ALL,”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, lymphoblastic leukemia, myelogenous leukemia, lymphocytic leukemia, myelocytic leukemia, Hodgkin's disease, non-Hodgkin's Lymphoma, Waldenstrom's macroglobulinemia, Heavy chain disease, and Polycythemia vera.

The invention encompasses kits to be used in assessing the expression of a particular RNA in a sample from a subject to assess the risk of developing disease. Kits include any combination of components that facilitates the performance of an assay. A kit that facilitates assessing the expression of an RNA may include suitable nucleic acid-based and immunological reagents as well as suitable buffers, control reagents, and printed protocols.

Kits that facilitate nucleic acid based methods may further include one or more of the following: specific nucleic acids such as oligonucleotides, labeling reagents, enzymes including PCR amplification reagents such as Taq or Pfu; reverse transcriptase, or one or more other polymerases, and/or reagents that facilitate hybridization. Specific nucleic acids may include nucleic acids, polynucleotides, oligonucleotides (DNA, or RNA), or any combination of molecules that includes one or more of the above, or any other molecular entity capable of specific binding to a nucleic acid biomarker. In one aspect of the invention, the specific nucleic acid comprises one or more oligonucleotides capable of hybridizing to the biomarker.

A specific nucleic acid may include a label. A label may be any substance capable of aiding a machine, detector, sensor, device, or enhanced or unenhanced human eye from differentiating a sample that that displays positive expression from a sample that displays reduced expression. Examples of labels include but are not limited to: a radioactive isotope or chelate thereof, a dye (fluorescent or nonfluorescent,) stain, enzyme, or nonradioactive metal. Specific examples include but are not limited to: fluorescein, biotin, digoxigenin, alkaline phosphatase, biotin, streptavidin, ³H, ¹⁴C, ³²P, ³⁵S, or any other compound capable of emitting radiation, rhodamine, 4-(4′-dimethylaminophenylazo) benzoic acid (“Dabcyl”); 4-(4′-dimethylamino-phenylazo)sulfonic acid (sulfonyl chloride) (“Dabsyl”); 5-((2-aminoethyl)-amino)-naphtalene-1-sulfonic acid (“EDANS”); Psoralene derivatives, haptens, cyanines, acridines, fluorescent rhodol derivatives, cholesterol derivatives; ethylenediaminetetraaceticacid (“EDTA”) and derivatives thereof or any other compound that signals the presence of the labeled nucleic acid. In one embodiment of the invention, the label includes one or more dyes optimized for use in genotyping. Examples of such dyes include but are not limited to: FAM, dR110, 5-FAM, 6FAM, dR6G, JOE, HEX, VIC, TET, dTAMRA, TAMRA, NED, dROX, PET, BHQ+, Gold540, and LIZ.

An oligonucleotide is a reagent capable of binding a nucleic acid sequence. An oligonucleotide may be any polynucleotide of at least 2 nucleotides. Oligonucleotides may be less than 10, less than 15, less than 20, less than 30, less than 40, less than 50, less than 75, less than 100, less than 200, less than 500, or more than 500 nucleotides in length. While oligonucleotides are often linear, they may, depending on their sequence and conditions, assume a two- or three-dimensional structure. Oligonucleotides may be chemically synthesized by any of a number of methods including sequential synthesis, solid phase synthesis, or any other synthesis method now known or yet to be disclosed. Alternatively, oligonucleotides may be produced by recombinant DNA based methods. One skilled in the art would understand the length of oligonucleotide necessary to perform a particular task. Oligonucleotides may be directly labeled, used as primers in PCR or sequencing reactions, or bound directly to a solid substrate as in oligonucleotide arrays.

A nucleotide is an individual deoxyribonucleotide or ribonucleotide base. Examples of nucleotides include but are not limited to: adenine, thymine, guanine, cytosine, and uracil, which may be abbreviated as A, T, G, C, or U in representations of oligonucleotide or polynucleotide sequence. Any molecule of two or more nucleotide bases, whether DNA or RNA, may be termed a nucleic acid.

When a nucleic acid such as an oligonucleotide includes a particular sequence, the sequence may be a part of a longer nucleic acid or may be the entirety of the sequence. The nucleic acid may contain nucleotides 5′ of the sequence, 3′ of the sequence, or both. The concept of a nucleic acid including a particular sequence further encompasses nucleic acids that contain less than the full sequence that are still capable of specifically detecting an allele. Nucleic acid sequences may be identified by the IUAPC letter code which is as follows: A—Adenine base; C—Cytosine base; G—guanine base; T or U—thymine or uracil base. M—A or C; R—A or G; W—A or T; S—C or G; Y—C or T; K—G or T; V—A or C or G; H—A or C or T; D—A or G or T; B—C or G or T; N or X—A or C or G or T. Note that T or U may be used interchangeably depending on whether the nucleic acid is DNA or RNA. A sequence having less than 60%, 70%, 80%, 90%, 95%, 99% or 100% identity to the identifying sequence may still be encompassed by the invention if it is able of binding to its complimentary sequence and/or facilitating nucleic acid amplification of a desired target sequence. If a sequence is represented in degenerate form; for example through the use of codes other than A, C, G, T, or U; the concept of a nucleic acid including the sequence also encompasses a mixture of nucleic acids of different sequences that still meet the conditions imposed by the degenerate sequence.

An oligonucleotide used to detect to an allele may be affixed to a solid substrate. Alternatively, the sample may be affixed to a solid substrate and the nucleic acid reagent placed into a mixture. For example, the nucleic acid reagent may be bound to a substrate in the case of an array or the sample may be bound to a substrate as the case of a Southern Blot, Northern blot or other method that affixes the sample to a substrate. A nucleic acid reagent or sample may be covalently bound to the substrate or it may be bound by some non covalent interaction including electrostatic, hydrophobic, hydrogen bonding, Van Der Waals, magnetic, or any other interaction by which an oligonucleotide may be attached to a substrate while maintaining its ability to recognize the allele to which it has specificity. A substrate may be any solid or semi solid material onto which a probe may be affixed, attached or printed, either singly or in the formation of a microarray. Examples of substrate materials include but are not limited to polyvinyl, polysterene, polypropylene, polyester or any other plastic, glass, silicon dioxide or other silanes, hydrogels, gold, platinum, microbeads, micelles and other lipid formations, nitrocellulose, or nylon membranes. The substrate may take any shape, including a spherical bead or flat surface.

In some aspects of the invention, the probe may be affixed to a solid substrate. In other aspects of the invention, the sample may be affixed to a solid substrate. A probe or sample may be covalently bound to the substrate or it may be bound by some non covalent interaction including electrostatic, hydrophobic, hydrogen bonding, Van Der Waals, magnetic, or any other interaction by which a probe such as an oligonucleotide probe may be attached to a substrate while maintaining its ability to recognize the allele to which it has specificity. A substrate may be any solid or semi solid material onto which a probe may be affixed, attached or printed, either singly or in the formation of a microarray. Examples of substrate materials include but are not limited to polyvinyl, polysterene, polypropylene, polyester or any other plastic, glass, silicon dioxide or other silanes, hydrogels, gold, platinum, microbeads, micelles and other lipid formations, nitrocellulose, or nylon membranes. The substrate may take any form, including a spherical bead or flat surface. For example, the probe may be bound to a substrate in the case of an array. The sample may be bound to a substrate as (for example) the case of a Southern Blot, Northern blot or other method that affixes the sample to a substrate.

A kit may also contain an indication of a result of the use of the kit that signifies a particular physiological or cellular characteristic. An indication includes any guide to a result that would signal the presence or absence of any physiological or cellular state that the kit is configured to predict. For example, the indication may be expressed numerically, expressed as a color or density of a color, expressed as an intensity of a band, derived from a standard curve, or expressed in comparison to a control. The indication may be communicated through the use of a writing that may be contained physically in or on the kit (on a piece of paper for example), posted on the Internet, mailed to the user separately from the kit, or embedded in a software package. The writing may be in any medium that communicates how the result may be used to predict the cellular or physiological characteristic such as a printed document, a photograph, sound, color, or any combination thereof.

The invention encompasses the detection of microRNA (that may be interchangeably be referred to as miRNA or miR) biomarkers and using the expression of the biomarkers to predict disease outcome.

MicroRNA has been shown to be a major new class biomolecules involved in control of gene expression. For example, in human heart, liver or brain, miRNA play a role in tissue specification or cell lineage decisions. In addition, miRNAs influence a variety of processes, including early development, cell proliferation and cell death, and apoptosis and fat metabolism. The large number of miRNA genes, the diverse expression patterns and the abundance of potential miRNA targets suggest that miRNAs may be a significant but unrecognized source of human genetic disease. Differences in miRNA expression have also been found to be associated with cancer diagnosis, prognosis, and susceptibility to treatments.

A mature miRNA is typically an 18-25 nucleotide non-coding RNA that regulates expression of mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNA's may also bind to target mRNAs and mediate gene silencing through the RNAi pathway. MiRNAs may also regulate gene expression by causing chromatin condensation.

Endogenously expressed miRNAs are processed by endonucleolytic cleavage from larger double-stranded RNA precursor molecules. The resulting small single-stranded miRNAs are incorporated into a multiprotein complex, termed RISC. The small RNA in RISC provides sequence information that is used to guide the RNA-protein complex to its target RNA molecules. The degree of complimentarily between the small RNA and its target determines the fate of the bound mRNA. Perfect pairing induces target RNA cleavage, as is the case for siRNAs and most plant miRNAs. In comparison, the imperfect pairing in the central part of the duplex leads to a block in translation.

MicroRNAs regulate various biological functions including developmental processes, developmental timing, cell proliferation, neuronal gene expression and cell fate, apoptosis, tissue growth, viral pathogenesis, brain morphogenesis, muscle differentiation, stem cell division and progression of human diseases. Many miRNAs are conserved in sequence and function between distantly related organisms. However, condition-specific, time-specific, and individual-specific levels of gene expression may be due to the interactions of different miRNAs which lead to genetic expression of various traits. The large number of miRNA genes, the diverse expression patterns and the abundance of potential miRNA targets suggest that miRNAs may be a significant but unrecognized source of human genetic diseases. MicroRNA genetic alterations, such as deletion, insertion, reversion or conversion, may affect the accuracy of miRNA related gene regulation. MicroRNA genetic alterations may be used as biomarkers for disease prognosis and diagnosis. Common methods of analyzing miRNA such as array-based methods are unable to detect mutated miRNA.

MicroRNA is readily detectable in blood and blood compartments such as serum or plasma or whole blood by any of a number of methods. See, for example, Chen X et al, Cell Research 18 983-984, October 2008; hereby incorporated by reference in its entirety.

MicroRNA may be amplified by any of a number of techniques including reverse transcription followed by PCR. Some techniques of reverse transcription of miR use a targeted stem-loop primer to prime reverse transcription of the miR into a cDNA template. The cDNA template may then be used as a primer for any type of PCR including any type of quantitative PCR. A stem-loop oligonucleotide is a single stranded oligonucleotide that includes a sequence capable of binding to a specific biomarker because it includes a nucleic acid sequence complementary to the biomarker. The sequence complementary to the biomarker is flanked by inverted repeats that form self-complementary sequences. Such nucleotides may contain a fluorophore quencher pair at the 5′ and 3′ ends of the oligonucleotide. (See Buzdin and Lukyanov in Nucleic Acids Hybridization Modern Applications, pp 85-96, Springer 2007, hereby incorporated by reference in its entirety.)

The invention encompasses methods of treating a patient based on the cohort in which the patient is classified. This includes the administration of one or more pharmaceutical compositions.

Methods of administration of a pharmaceutical composition include, but are not limited to, oral administration and parenteral administration. Parenteral administration includes, but is not limited to intradermal, intramuscular, intraperitoneal, intravenous, subcutaneous, intranasal, epidural, sublingual, intramsal, intracerebral, iratraventricular, intrathecal, intravaginal, transdermal, rectal, by inhalation, or topically to the ears, nose, eyes, or skin. Other methods of administration include but are not limited to infusion techniques including infusion or bolus injection, by absorption through epithelial or mucocutaneous linings such as oral mucosa, rectal and intestinal mucosa. Compositions for parenteral administration may be enclosed in ampoule, a disposable syringe or a multiple-dose vial made of glass, plastic or other material.

Administration may be systemic or local. Local administration is administration a pharmaceutical composition to an area in need of treatment. Examples include local infusion during surgery; topical application, by local injection; by a catheter; by a suppository; or by an implant. Administration may be by direct injection at the site (or former site) of a cancer, tumor, or precancerous tissue or into the central nervous system by any suitable route, including intraventricular and intrathecal injection. Intraventricular injection can be facilitated by an intraventricular catheter, for example, attached to a reservoir, such as an Ommaya reservoir. Pulmonary administration may be achieved by any of a number of methods known in the art. Examples include use of an inhaler or nebulizer, formulation with an aerosolizing agent, or via perfusion in a fluorocarbon or synthetic pulmonary surfactant. The disclosed compound may be delivered in the context of a vesicle such as a liposome or any other natural or synthetic vesicle.

Addition of a pharmaceutical composition to cancer cells includes all actions by which an effect of the pharmaceutical composition on the cancer cell is realized. The type of addition chosen will depend upon whether the cancer cells are in vivo, ex vivo, or in vitro, the physical or chemical properties of the pharmaceutical composition, and the effect the composition is to have on the cancer cell. Nonlimiting examples of addition include addition of a solution including the pharmaceutical composition to tissue culture media in which in vitro cancer cells are growing; any method by which a pharmaceutical composition may be administered to an animal including intravenous, per os, parenteral, or any other of the methods of administration; or the activation or inhibition of cells that in turn have effects on the cancer cells such as immune cells (e.g. macophages and CD8+ T cells) or endothelial cells that may differentiate into blood vessel structures in the process of angiogenesis or vasculogenesis.

Treatment of a condition is the practice of any method, process, or procedure with the intent of halting, inhibiting, slowing or reversing the progression of a disease, disorder or condition, substantially ameliorating clinical symptoms of a disease disorder or condition, or substantially preventing the appearance of clinical symptoms of a disease, disorder or condition, up to and including returning the diseased entity to its condition prior to the development of the disease. Treatment is contemplated in living entities including but not limited to mammals (particularly humans) as well as other mammals of economic or social importance, including those of an endangered status. Further examples include livestock or other animals generally bred for human consumption and domesticated companion animals. A patient includes any human being, nonhuman primate, companion animal, or mammal suffering from a disease.

Pharmaceutical compositions may be administered prior to, concurrently with, or after administration of a second pharmaceutical composition. If the compositions are administered concurrently, they are administered within one minute of each other, including multiple compositions that are part of the same formulation. If not administered concurrently, the second pharmaceutical composition may be administered a period of one or more minutes, hours, days, weeks, or months before or after the pharmaceutical composition that includes the compound Alternatively, a combination of pharmaceutical compositions may be cyclically administered. Cycling therapy involves the administration of one or more pharmaceutical compositions for a period of time, followed by the administration of one or more different pharmaceutical compositions for a period of time and repeating this sequential administration, in order to reduce the development of resistance to one or more of the compositions, to avoid or reduce the side effects of one or more of the compositions, and/or to improve the efficacy of the treatment.

The treatment of cancer includes the prevention of progression of the cancer to a neoplastic, malignant or metastatic state. Such preventative use is indicated in conditions known or suspected of preceding progression to cancer, in particular, where non- or precancerous cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-90, incorporated by reference). Hyperplasia is a form of controlled cell proliferation involving an increase in cell number in a tissue or organ, without significant alteration in structure or activity. For example, endometrial hyperplasia often precedes endometrial cancer and precancerous colon polyps often transform into cancerous lesions. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype or of a malignant phenotype, displayed in vivo or displayed in vitro by a cell sample derived from a patient can indicate the desirability of prophylactic/therapeutic administration of the pharmaceutical composition that includes the compound. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. Further examples include leukoplakia, featuring a benign-appearing hyperplastic or dysplastic lesion of the epithelium, or Bowen's disease, a carcinoma in situ. Both of theses are pre-cancerous lesions indicative of the desirability of prophylactic intervention. In another example, fibrocystic disease including cystic hyperplasia, mammary dysplasia, adenosis, or benign epithelial hyperplasia is indicates desirability of prophylactic intervention.

A subject or patient may be suspected of having cancer by any of a number of diagnostic signs including unexplained weight loss, fever, fatigue, pain, changes in skin color or texture, changes in elimination, sores that fail to heal, patches inside the mouth unusual bleeding, a thickening or lump on some part of the body, detectable either externally or internally, indigestion or trouble swallowing, changes in warts or moles, or prolonged coughing. Additional factors such as age, family history, environmental exposure may be factors in suspecting that a person has cancer. Additionally, CAT scans, MRI's or other imaging technologies may be used to see internal tumors. A subject or patient may be suspected of having small cell lung cancer by any of a number of symptoms, including but not limited to the presence of a persistent cough, blood in sputum, wheezing, chest pains, unexplained weight loss, fever that is independent of an infection, or swelling of the face. Knowing that a patient has a type of cancer is included in the definition of suspecting that a patient has that type of cancer in that if the patient is known to have the particular cancer, the patient is by definition suspected of having the type of cancer.

Use of pharmaceutical compositions may be determined by one or more physical factors such as tumor size and grade or one or more molecular biomarkers and/or expression signatures that indicate prognosis and the likely response to treatment with the compound. For example, determination of estrogen (ER) and progesterone (PR) steroid hormone receptor status has become a routine procedure in assessment of breast cancer patients. See, for example, Fitzgibbons et al, Arch. Pathol. Lab. Med. 124:966-78, 2000, incorporated by reference. Tumors that are hormone receptor positive are more likely to respond to hormone therapy and also typically grow less aggressively, thereby resulting in a better prognosis for patients with ER+/PR+ tumors. In a further example, overexpression of human epidermal growth factor receptor 2 (HER-2/neu), a transmembrane tyrosine kinase receptor protein, has been correlated with poor breast cancer prognosis (see, e.g., Ross et al, The Oncologist 8:307-25, 2003), and Her-2 expression levels in breast tumors are used to predict response to the anti-Her-2 monoclonal antibody therapeutic trastuzumab (Herceptin®, Genentech, South San Francisco, Calif.).

In the treatment of cancer, the diseased entity may exhibit one or more predisposing factors for malignancy that may be treated by administration of a pharmaceutical composition. Such predisposing factors include but are not limited to chromosomal translocations associated with a malignancy such as the Philadelphia chromosome for chronic myelogenous leukemia and t (14; 18) for follicular lymphoma; an incidence of polyposis or Gardner's syndrome that are indicative of colon cancer; benign monoclonal gammopathy which is indicative of multiple myeloma, kinship with persons who have had or currently have a cancer or precancerous disease, exposure to carcinogens, presence or absence of one or more biomarkers associated with cancer, or any other predisposing factor that indicates in increased incidence of cancer now known or yet to be disclosed.

Treatment of cancer further encompasses methods that comprise therapies that include the administration of a pharmaceutical composition in combination with another treatment modality. Such treatment modalities include but are not limited to, radiotherapy, chemotherapy, surgery, immunotherapy, cancer vaccines, radioimmunotherapy, treatment with other pharmaceutical compositions, or any other method that effectively treats cancer in combination with the disclosed compound now known or yet to be disclosed. Combination therapies may act synergistically. That is, the combination of the two therapies is more effective than either therapy administered alone. This results in a situation in which lower dosages of each treatment modality may be used effectively. This in turn reduces the toxicity and side effects, if any, associated with the administration either modality without a reduction in efficacy.

A pharmaceutical composition may be administered in combination with a therapeutically effective amount of radiotherapy. The radiotherapy may be administered concurrently with, prior to, or following the administration of the pharmaceutical composition including the compound. The radiotherapy may act additively or synergistically with the pharmaceutical composition including the compound. This particular aspect of the invention would be most effective in cancers known to be responsive to radiotherapy. Cancers known to be responsive to radiotherapy include, but are not limited to, Non-Hodgkin's lymphoma, Hodgkin's disease, Ewing's sarcoma, testicular cancer, prostate cancer, ovarian cancer, bladder cancer, larynx cancer, cervical cancer, nasopharynx cancer, breast cancer, colon cancer, pancreatic cancer, head and neck cancer, esophogeal cancer, rectal cancer, small-cell lung cancer, non-small cell lung cancer, brain tumors, other CNS neoplasms, or any other such tumor now known or yet to be disclosed.

Examples of pharmaceutical compositions that may be used in combination may include nucleic acid binding compositions such as cis-diamminedichloro platinum (II) (cisplatin), doxorubicin, 5-fluorouracil, taxol, and topoisomerase inhibitors such as etoposide, teniposide, irinotecan and topotecan. Still other pharmaceutical compositions include antiemetic compositions such as metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron.

Still other examples of pharmaceutical compositions that may be used in combination are hematopoietic colony stimulating factors. Examples of hematopoietic colony stimulating factors include, but are not limited to, filgrastim, sargramostim, molgramostim and epoietin alfa. Alternatively, the pharmaceutical composition including the disclosed compound may be used in combination with an anxiolytic agent. Examples of anxiolytic agents include, but are not limited to, buspirone, and benzodiazepines such as diazepam, lorazepam, oxazapam, chlorazepate, clonazepam, chlordiazepoxide and alprazolam.

Pharmaceutical compositions that may be used in combination with pharmaceutical compositions that include the disclosed compound may include analgesic agents. Such agents may be opioid or non-opioid analgesic. Non-limiting examples of opioid analgesics include morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam, sulindac or any other analgesic now known or yet to be disclosed.

In other aspects of the invention, pharmaceutical compositions may be used in combination with a method that involves treatment of cancer ex vivo. One example of such a treatment is an autologous stem cell transplant. In this method, a diseased entity's autologous hematopoietic stem cells are harvested and purged of all cancer cells. A therapeutic amount of a pharmaceutical composition including the disclosed compound may then be administered to the patient prior to restoring the entity's bone marrow by addition of either the patient's own or donor stem cells.

If a patient is classified into a cohort likely to respond to systemic chemotherapy, then, treatment might comprise the administration of a pharmaceutical composition appropriate for use in systemic chemotherapy. Systemic chemotherapy is the administration of any substance that may be dispersed throughout the body in order to affect any cancer cell in any location in the patient. Systemic chemotherapy agents include but need not be limited to cisplatin, carboplatin, etoposide, ironectan, topotecan, cyclophosphamide, doxorubicin, vincristine, amrubicin, epirubicin, or S-1 administered alone or in combination with each other or any other pharmaceutical composition, or any other systemic chemotherapy agent now known or yet to be disclosed or discovered.

Tumors that are resistant to systemic chemotherapy might be interchangeably referred to as chemoresistant. There are two different classes of chemoresistant tumors. One type is intrinsically chemoresistant—that is, chemoresistance is an inherent quality of the tumor that the tumor possesses intrinsic factors that render it resistant to chemotherapy. Another type of chemoresistance is acquired chemoresistance—that is, chemoresistance is resistance that the tumor develops in response to treatment with, chemotherapeutic agent.

If a patient is classified into a cohort unlikely to respond to systemic chemotherapy, then treatment might comprise the administration of a pharmaceutical composition specifically targeted to tumors that are resistant to systemic chemotherapy. Examples of such pharmaceutical compositions may include but need not be limited to monoclonal antibodies or small molecules that block CD9 activity, siRNA that block the expression of CD9, chemokine CXCL12 agonists, antibodies or small molecules that block fibronectin β1 integrin (Kohmo S et al, Cancer Res 70, 8025-8035, (October 2010)); FGFR inhibitors such as PD 173074 (Pardo O E et al, Cancer Res 69, 8645-8651 (November 2009)); xc-cysteine transporter inhibitors such as monosodium glutamate or sulfasalazine (Guan J et al, Cancer Chemother Pharmacol 64, 463-472 (2008)); urokinase plasminogen activator (uPA) inhibitors (Gutova et al, PLoS One 2 e243, 10.1371/journal.pone0000243 (2007)) such as 2-pyridinylguanidines and WX-UK1, ATP binding cassette (ABC) transporter inhibitors (Dean M et al, Nat Rev Cancer 5, 275-284 (2005)), a tumor vaccine, or any other pharmaceutical composition capable of affecting chemoresistant non-small cell lung cancer now known or yet to be discovered or disclosed.

Cancers that may be treated by pharmaceutical compositions include solid tumors such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Additional cancers that may be treated by pharmaceutical compositions include blood borne cancers such as acute lymphoblastic leukemia (“ALL,”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, lymphoblastic leukemia, myelogenous leukemia, lymphocytic leukemia, myelocytic leukemia, Hodgkin's disease, non-Hodgkin's Lymphoma, Waldenstrom's macroglobulinemia, Heavy chain disease, and Polycythemia vera.

### Example

### MiRNA Biomarkers Predictive of Outcome in Small Cell Lung Cancer

Elements and acts in the example are intended to illustrate the invention for the sake of simplicity and have not necessarily been rendered according to any particular sequence or embodiment. The example is further intended to establish possession of the invention by the Inventor.

Tumor samples were obtained after prior approval of the local Institutional Review Board (IRB) from patients diagnosed between the period 2001 to 2007 and receiving care and follow-up at Scottsdale Healthcare (Scottsdale, Ariz.). All patients subsequently received systemic chemotherapy. Clinical characteristics included age at diagnosis, gender, SCLC histology, limited- or extensive-stage disease, smoking history, and statin use. The presence of baseline co-morbidities including: cardiovascular disease (CAD), lung disease [chronic obstructive pulmonary disease (COPD) or emphysema], thrombotic event (deep vein thrombosis or pulmonary embolism), diabetes, hypertension, peripheral vascular disease (PVD), and hyperlipidemia were also available. RNA extraction and miRNA microarray profiling was then performed.

Tumor cells were manually scraped from formalin-fixed, paraffin embedded (FFPE) SCLC tumor samples previously sectioned and mounted on slides. The samples were de-paraffinized in xylene at 50° C. Three to nine sections per tumor sample were collected. Samples were then centrifuged. The pellet was then washed in 100% ethanol. After removal of the ethanol, the pellet was treated with proteinase K in an appropriate buffer at 50° C. for 3 hours. Total RNA was extracted from the resulting solution using a TRI reagent. Total RNA was then eluted in DEPC water. The concentration and purity of isolated RNA was then estimated using a NanoDrop microspectrophotometer. Samples from which 1 μg of total RNA was isolated were hybridized to the GenoExplorer microRNA Expression System (GenoSensor Corp, Tempe Ariz.), which is an miRNA microarray platform containing probes in triplicate for 880 validated human mature miRNAs with an additional 473 validated human pre-miRNAs (Sanger miRNA Registry, version 13.0 Mar. 2009, www.mirbase.org) along with positive and negative control probes.

The signal intensities for each miRNA detected on the microarray profiling platform were normalized by sequentially dividing by the mean signal intensities of housekeeping gene probes: RNU6 and 5S-rRNA. miRNA analysis was performed using XenoBase version v3.4.2009.0922, a data integration and discovery tool developed at the Van Andel Research Institute. XenoBase: A personalized medicine tool for integration of clinical, genomic, and laboratory data. Van Andel Research Institute, Grand Rapids Mich., USA. Xenobase was used to integrate the clinical data, miRNA probe data, and interaction data from the Sanger miRNA Registry Version 14.0 (September 2009). Confirmation of the top 16 miRNA biomarker candidates from the microarray was performed by quantitative reverse transcription PCR (qRT-PCR) analysis. Quantitative real-time PCR (qRT-PCR) was performed using the total RNA extracted from these samples run in triplicate on a 384-well plate and normalized to 5S-rRNA and RNU6. The GenoExplorer miRNA First-strand cDNA Core Kit (GenoSensor Corporation, Tempe Ariz.) was used to generate miRNA first-strand cDNA. miRNA expression was measured using an miRNA specific forward primer and a universal reverse primer. Expression was measured by SYBR green. PCR reaction volumes were 15 μl. Reaction conditions included a 15 minute denaturation at 94° C. followed by 45 cycles of 94° C. for 30 seconds, 59° C. for 15 seconds, and 72° C. for 30 seconds. Melting curve analysis was used to assess the specificity of the amplified product.

The miRNA microarray expression data was stratified into groups based on survival time and chemoresistance (defined as disease progression by clinical or first radiologic assessment). The top 16 individual miRNAs that were significant by p-value in the array analysis were selected for validation with qRT-PCR. Expression level of the top 16 miRNA candidates for chemoresistance were assessed for validation by qRT-PCR normalized to RNU6 and 5S-rRNA. Fisher's exact test was used to identify any significant (p<0.05) associations between baseline co-morbidities and chemoresistance.

To facilitate the group wise analysis, Kaplan Meier plots, and clustering analysis, (R version 2.10.0) was used to analyze the data using both univariate and multivariate Cox proportional hazards models. Univariate Cox proportional hazards models were used to examine each of the clinical factors and qRT-PCR miRNA values for significance. Those clinical factors and miRNAs that were significant were included in a multivariate Cox proportional hazard model. A reduced data set which included only subjects with no missing qRTPCR data (N=23) and the same factors as the multivariate analysis was analyzed using a stepwise procedure which included both forward and backward selection methods. All the Cox proportional hazards analyses were performed using R version 2.10.0. Group wise analysis using t-test, Kaplan Meier plots and clustering were performed using XenoBase v3.4.2009.0922.

Total RNA was extracted from 34 FFPE SCLC tumor specimens. All 34 samples had sufficient total RNA yield to perform miRNA microarray profiling, while 28 samples had sufficient total RNA for qRT-PCR. MiRNA profiling data from all 34 cases that exceeded positive control thresholds for RNU6 and 5S-rRNA were subsequently analyzed using the XenoBase system. Quantitative RT-PCR results of the 16 miRNA candidates that were most likely to be biomarkers for chemoresistance were analyzed on the 28 samples with available RNA. Baseline characteristics and survival data for the 34 SCLC cases are shown in Table 1.

TABLE 1
Patient and Disease characteristics

| Clinical Factors | Results | |
|---|---|---|
| Median Age years (range) (N = 34) | 69.09 | (42.52-82.48) |
| Gender (%) (N = 34) | | |
| Male | 17 | (50%) |
| Female | 17 | (50%) |
| Disease stage (N = 33) | | |
| Limited (%) | 4 | (12.1%) |
| Extensive-stage (%) | 29 | (87.9%) |
| Baseline co-morbidities (N = 34) | | |
| Coronary artery disease | 7 | (20.6%) |
| Lung disease | 11 | (32.4%) |
| Deep vein thrombosis | 2 | (5.9%) |
| Diabetes | 5 | (14.7%) |
| Hypertension | 16 | (47.1% |
| Peripheral vascular disease | 3 | (8.8%) |
| Hyperlipidemia | 9 | (26.5%) |
| Cigarette pack/year (N = 25) | | |
| Median (range) | 40 | (13-165) |
| Chemotherapy (N = 34) | | |
| Cisplatin-containing regimen | 10 | (29.4%) |
| Carboplatin-containing regimen | 18 | (52.9%) |
| Other | 6 | (17.6%) |
| Received radiation during first-line therapy | 16 | (47.1%) |
| (N = 16) | | |
| Response (N = 21) | | |
| Complete response | 2 | (9.5%) |
| Partial response | 13 | (61.9%) |
| Stable disease | 2 | (9.5%) |
| Progressive disease | 4 | (19.1%) |
| Median Survival in days (range) (N = 34) | 246.5 | (3-2384) |

The median age was 69.09 years (range 42.52-82.48). There were 4 (12.1%) limited-stage and 29 (87.9%) extensive-stage patients at diagnosis.

Of the top 16 miRNA biomarker candidates for chemoresistance by miRNA microarray analysis, (Table 2) three miRNAs were significantly differentially expressed by qRT-PCR, including miR-92a-2* (p=0.010), miR-147 (p=0.018), and miR-574-5p (p=0.039). (Table 3).

TABLE 2
Top 16 candidate biomarkers from microarray.

| | Surviv- | Surviv- | | |
|---|---|---|---|---|
| | al ≦80 | al ≧270 | Fold | |
| miRNA name | days | days | Change | p-value |
| hsa-mir-198 | 186.0416667 | 154.3095238 | 0.82943529 | 0.005109 |
| hsa-miR-92a-2* | 154.6666667 | 132.9285714 | 0.85945197 | 0.006161 |
| hsa-miR-206 | 168.8333334 | 133.8095238 | 0.792553942 | 0.00979 |
| hsa-miR-147 | 158.125 | 130.2857143 | 0.823941276 | 0.012193 |
| hsa-miR-585 | 152.0833333 | 127.8095238 | 0.84039139 | 0.012262 |
| hsa-miR-92b* | 421.6666667 | 249.1190476 | 0.59079616 | 0.012929 |
| hsa-miR-574-5p | 723.2083333 | 219.0238095 | 0.302850229 | 0.015218 |
| hsa-miR-574-3p | 427.7916667 | 172.3809524 | 0.402955377 | 0.015539 |
| hsa-miR-631 | 157.9583334 | 131.5952381 | 0.833100953 | 0.015919 |
| hsa-miR-744 | 247.75 | 188.0714286 | 0.759117774 | 0.016603 |
| hsa-miR-885-5p | 159.1666667 | 134.6904762 | 0.846222887 | 0.01696 |
| hsa-miR-936 | 185 | 144.3571429 | 0.78030888 | 0.01722 |
| hsa-miR-765 | 301.9583333 | 192.0476191 | 0.636007018 | 0.01971 |
| hsa-miR-1266 | 161.9583333 | 132.5 | 0.818111654 | 0.020012 |
| hsa-miR-1275 | 423.0833333 | 221.9047619 | 0.524494218 | 0.020306 |

TABLE 3
miRNA significantly differentially expressed by qRT-PCR

| miRNA | p-value |
|---|---|
| miR-92a-2* | 0.010 |
| miR-147 | 0.018 |
| miR-574-5p | 0.039 |

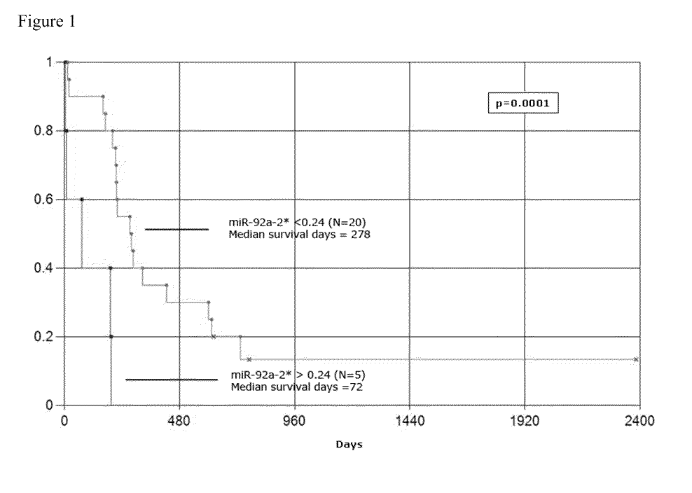

There were no significant associations between gender or baseline co-morbidities and chemoresistance. Across all miRNAs measured by qRT-PCR, expression levels were not significantly altered based on the biopsy location of the SCLC sample (data not shown). By univariate analysis, gender (p=0.012), CAD (p=0.036), and PVD (p=0.027) were significantly associated with survival. The miRNAs associated with survival by univariate analysis were miR-92a-2* (p=0.007), miR-147 (p=0.014), and miR-585 (p=0.031). There were no significant associations between baseline co-morbidities and these three miRNAs. Multivariate analysis using a Cox proportional hazard model was performed using stepwise selection for the following factors that showed significance by univariate analysis: CAD, PVD, gender, miR-92a-2*, miR-147, and miR-585. MiR-92a-2* expression contributed significantly to survival (p=0.015). FIG. 1 displays the Kaplan-Meier survival curve for miR-92a-2*, illustrating expression levels less than 0.24 (normalized to RNU6 and 5S-rRNA) is associated with significantly improved median survival compared to expression levels greater than 0.24 (log-rank p value=0.0001). These tumor miRNAs are therefore predictive biomarkers for chemoresistance and prognostic biomarkers for survival for SCLC patients treated with systemic chemotherapy.

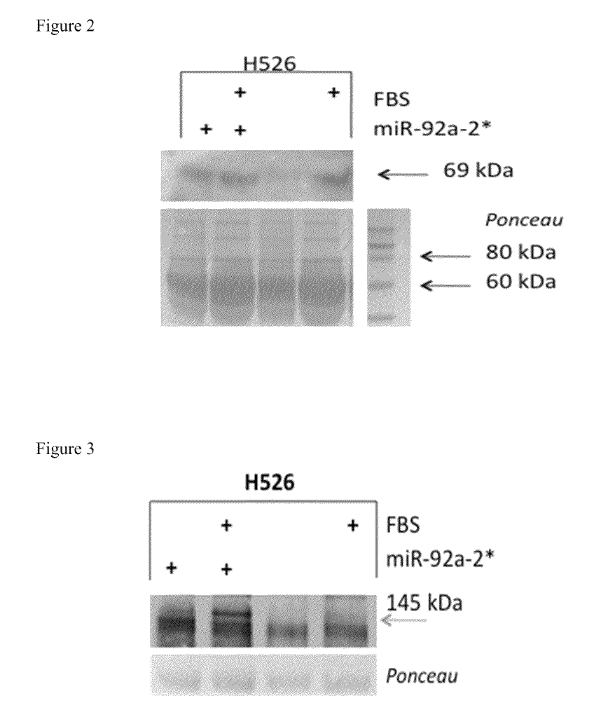

Hepatocyte growth factor (HGF) is known to activate c-MET in SCLC. In turn c-MET phosphorylation is known to induce platinum resistance in lung cancer (see References 8 and 9). Both miR-92a-2* and miR-147 overexpression in SCLC cell lines increases chemoresistance to cisplatin and etoposide combination treatment in vitro. FIGS. 2 and 3 demonstrate that miR-92a-2* overexpression confers chemoresistance upon SCLC cell lines.

Referring now to FIG. 2, which depicts increased secreted HGF in H526 overexpressing miR-92a-2* (H526-92) SCLC cells. Media were collected from serum starved and unstarved (FBS+) H526 engineered to overexpress miR-92a-2* (H526-92) and H526 empty vector cells (as indicated on the figure without a + for miR-92a-2*). Media were dialyzed and immunoblotted. The immunoblot shows increased HGF (69 kDa) in serum starved H526 cells compared to serum starved H526-empty vector cells. That there is no discernible difference in the serum unstarved condition, confirms increased HGF from H526-92 cells. Ponceau staining shows equal loading. (FBS: Fetal Bovine Serum).

Referring now to FIG. 3, which depicts increased phospho-MET in H526-92 SCLC cells. Lysates were collected from serum starved and unstarved (FBS+) H526-92 and H526 empty vector cells. After immunoprecipitating for phospho-tyrosine, the membrane was immunoblotted with an antibody specific to c-MET. This immunoblot shows increased phospho-MET (145 kDa) in H526-92 in both the serum starved and unstarved conditions relative to H526-empty cells. Ponceau staining shows equal loading. (FBS: Fetal Bovine Serum).

### REFERENCES

So as to reduce the complexity and length of the Detailed Specification, Inventors herein expressly incorporate by reference all of the following materials to the extent allowed.
1. Calin G A et al, Proc Natl Acad Sci USA 99, 15524-15529 (2002).
2. Kumar M S et al, Nat Genet 39, 673-677 (2007).
3. Eder M, et al, N Engl J Med; 352, 2446-2448 (2005).
4. Scott G K et al J Biol Chem 282, 1479-1486 (2007).
5. Calin G A, et al. Proc Natl Acad Sci USA; 101, 11755-11760 (2004).
6. Lu J et al. Nature 435, 834-838. (2005).
7. Brown et al. Publication Number US 2007/0161004, filed May 2005.
8. Ma P C et al, Br. J. Cancer 6, 368-377, (2007).
9. Chen J T et al, Am J Respir Cell Mol Bio 5, 559-565 (2008).
10. Colombo T et al, Cancer Chemother Pharmacol 7, 127-131 (1982).
11. Tang B C et al, Biomaterials 31, 339-344 (January 2010).
12. Harivardhan Reddy L et al, J Control Release 105, 185-198 (2005).
13. Morita M et al, Nippon Yakuigaku Zasshi 87, 53-66 (1986).
14. Kohmo S. et al, Cancer Res 70, 8025-8035, (October 2010).
15. Pardo O E et al, Cancer Res 69, 8645-8651 (November 2009).
16. Guan J et al, Cancer Chemother Pharmacol 64, 463-472 (2008).
17. Gutova et al, PLoS One 2, e243, 10.1371/journal.pone.0000243 (2007).
18. Dean M et al, Nat Rev Cancer 5, 275-284 (2005).
19. Jemal A et al, CA Cancer J Clin 59, 225-249 (2009).
20. Jackman D M and Johnson B E, Lancet 366, 1385-1396 (2005).
21. Sandler A B, Semin Oncol 30, 9-25 (2003).
22. Brock M V et al, N Engl J Med 358, 1118-1128 (2008).
23. Chen H Y et al, N Engl J Med 356, 11-20 (2007).
24. Potti A et al, N Engl J Med 355, 570-580 (2006).
25. Yanihara N et al, Cancer Cell 9, 189-198 (2006).
26. Miko E et al, Exp Lung Res 35, 646-664 (2009).
27. XenoBase: A personalized medicine tool for integration of clinical, genomic, and laboratory data. Van Andel Research Institute, Grand Rapids, Mich., USA.
28. R Development Core Team (2009). R: A language and environment for statistical computing, R Foundation for Statistical Computing, Vienna, Austria, ISBM 3-90051-07-0, www.R-project.org
29. Zheng T et al, Int J Cancer 126, 2-10 (January 2010).
30. Brambilia E et al, J Clin Oncol 9, 50-61 (1991).
31. Pfeiffer M et al, Br J Cancer 100, 1949-1956 (2009).
32. Rudin C M et al, J Clin Oncol 26, 870-876 (2008).
33. www.mirbase.org.
34. Landais S et al, Cancer Res 67, 5699-5707 (2007).

What is claimed is:

1. A method of classifying a lung cancer patient into a cohort, the method comprising:
adding a first reagent capable of binding to a first biomarker to a mixture comprising a nucleic acid isolated from a sample from the patient;
subjecting the mixture to conditions that allow detection of the binding of the first oligonucleotide to the biomarker; and
classifying the patient into a cohort on the basis of a result of the binding of the first oligonucleotide to the nucleic acid isolated from the sample;
wherein the first biomarker includes a sequence selected from the group consisting of SEQ ID NO. 1, SEQ ID NO. 2, and SEQ ID NO. 3; and
wherein the cohort is selected from the group consisting of a cohort of patients likely to respond to systemic chemotherapy and a cohort of patients unlikely to respond to systemic chemotherapy.

2. The method of claim 1 wherein the sample comprises a blood fraction selected from the group consisting of serum, plasma, and whole blood.

3. The method of claim 1 wherein the sample comprises tumor tissue.

4. The method of claim 4 wherein the patient is suspected of having small cell lung cancer.

5. The method of claim 1 wherein the first reagent comprises a first oligonucleotide.

6. The method of claim 5 wherein the first oligonucleotide is a stem-loop oligonucleotide.

7. The method of claim 6 further comprising adding a reverse transcriptase to the mixture and wherein the conditions comprise the synthesis of a reverse transcription product comprising the biomarker.

8. The method of claim 7 further comprising a second oligonucleotide and a third oligonucleotide to the mixture, wherein the second oligonucleotide and the third oligonucleotide each bind to part of the reverse transcription product and wherein the conditions further comprise nucleic acid amplification.

9. The method of claim 8 further comprising adding a fourth oligonucleotide to the mixture, wherein the fourth oligonucleotide is capable of binding to a sequence on the reverse transcription product between the sequences to which the second nucleic acid and the third nucleic acid are capable of binding.

10. The method of claim 9 wherein the fourth oligonucleotide comprises a fluorescent label.

11. The method of claim 10 wherein the fluorescent label is selected from the group consisting of FAM, dR110, 5-FAM, 6FAM, dR6G, JOE, HEX, VIC, TET, dTAMRA, TAMRA, NED, dROX, PET, BHQ+, Gold540, and LIZ.

12. The method of claim 7 further comprising performing nucleic acid sequencing on the reverse transcription product.

13. The method of claim 1 wherein the first reagent is affixed to a substrate.

14. The method of claim 13 wherein a second reagent capable of binding to a second biomarker is affixed to the substrate and wherein the substrate is configured to form a microarray.

15. A method of treating a lung cancer patient, the method comprising:
adding a first reagent capable of binding to a first biomarker to a mixture comprising a nucleic acid isolated from a sample from the patient;
subjecting the mixture to conditions that allow detection of the binding of the first oligonucleotide to the biomarker; and
treating the patient on the basis of a result of the binding of the first oligonucleotide to the nucleic acid;
wherein the first biomarker includes a sequence selected from the group consisting of SEQ ID NO. 1, SEQ ID NO. 2, and SEQ ID NO. 3.

16. The method of claim 15 wherein the lung cancer is small cell lung cancer.

17. The method of claim 15 wherein the result comprises expression below a threshold and wherein treating the patient comprises administration of systemic chemotherapy.

18. The method of claim 17 wherein the threshold comprises an expression level of 0.1 to 0.5 measured by quantitative reverse transcription PCR normalized to the expression of a housekeeping gene selected from the group consisting of SEQ ID NO. 4 and SEQ ID NO. 5.

19. The method of claim 17 wherein treatment comprises administration of a pharmaceutical composition comprising a drug selected from the group consisting of cisplatin, carboplatin, etoposide, ironectan, topotecan, cyclophosphamide, doxorubicin, vincristine, amrubicin, epirubicin, and S-1.

20. The method of claim 15 wherein the result comprises expression above a threshold and wherein treating the patient comprises administration of a pharmaceutical composition with an effect on a chemoresistant tumor.

21. The method of claim 20 wherein the threshold comprises an expression level of 0.1 to 0.5 measured by quantitative reverse transcription PCR normalized to the expression of SEQ ID NO. 4 and SEQ ID NO. 5.

22. The method of claim 20 wherein the pharmaceutical composition comprises a drug from a class selected from the group consisting CD9 inhibitors, chemokine CXCL12 agonists, fibronectin β1 integrin inhibitors, FGFR inhibitors, xc-cysteine transporter inhibitors, urokinase plasminogen activator (uPA) inhibitors, and ATP binding cassette (ABC) transporter inhibitors.

23. A kit used to classify a patient into a cohort, the kit comprising:
a first reagent capable of binding to a first biomarker represented by a sequence selected from the group consisting of SEQ ID NO. 1, SEQ ID NO. 2, and SEQ ID NO. 3; and
an indication of a result of the binding of the first biomarker to the sample;
wherein the indication signifies identification of the patient as belonging to the cohort; and wherein the cohort is selected from the group consisting of a cohort of patients likely to respond to systemic chemotherapy and a cohort of patients unlikely to respond to systemic chemotherapy.

24. The kit of claim 23 wherein the first reagent comprises a first oligonucleotide.

25. The kit of claim 24 wherein the first oligonucleotide is a stem loop oligonucleotide.

26. The kit of claim 24 further comprising a second oligonucleotide wherein the second oligonucleotide is capable of binding to a second biomarker, wherein the second biomarker comprises a housekeeping gene.

27. The kit of claim 26 wherein the second biomarker is selected from the group consisting of SEQ ID NO. 4 and SEQ ID NO. 5.

28. The kit of claim 23 further comprising an enzyme.

29. The kit of claim 28 wherein the enzyme is selected from the group consisting of: DNA polymerase, thermostable DNA polymerase, and reverse transcriptase.

30. The kit of claim 24 wherein the first oligonucleotide is affixed to a substrate.

31. The kit of claim 30 further comprising a second oligonucleotide affixed to the substrate and wherein the substrate is configured to form a microarray.

32. The kit of claim 23 wherein the result comprises an element selected from the group consisting of: a positive control, a numerical value, a Ct value, and a level of expression normalized to a housekeeping gene.

33. The kit of claim 23 wherein the indication comprises software configured to detect a level of expression as an input and classification of the subject into the cohort as an output.

34. The kit of claim 23 wherein the indication is physically included with the kit.

35. The kit of claim 23 wherein the indication is made available via a website.
